(19) United States
(12) Patent Application Publication
(10) Pub. No.: US 2012/0208232 A1
(43) Pub. Date: Aug. 16, 2012

(54) Cell-Free Synthesis of Active Reprogramming Transcription Factors

(76) Inventors: William Yang, Stanford, CA (US); Kedar Patel, Fremont, CA (US); Yohannes T. Ghebremariam, Stanta Clara, CA (US); Ji Eun Lee, Menlo Park, CA (US); Hann-Chung Wong, Lutherville, MD (US); John P. Cooke, Palo Alto, CA (US); James Robert Swartz, Menlo Park, CA (US)

(21) Appl. No.: 13/384,209

(22) PCT Filed: Jul. 14, 2010
(86) PCT No.: PCT/US10/41987

§ 371 Date: Apr. 25, 2012

(30) Foreign Application Priority Data
Jul. 15, 2009 (US) 61271000

(51) Int. Cl. C12P 21/00

(52) U.S. Cl. 435/68.1

(57) ABSTRACT

Compositions and methods are provided for the cell-free synthesis of active reprogramming factor polypeptides. The reprogramming factors may be synthesized as fusion proteins comprising a permeant domain, such as polyarginme. The cell free-synthesis may be conducted at about 25 C in a bacterial cell extract from genetically alterd cells having decreased endogenous protease activity Further, the proteins may comprise a fusion partner which enhances solubility and may be refolded on a column.

### BACKGROUND OF THE INVENTION

It has been shown that viral expression of a handful of human transactivating factors is capable of reprogramming human somatic cells into a pluripotent state. The iPSCs self-renew and differentiate into a wide variety of cell types, making them an appealing option for disease- and patient-specific regenerative medicine therapies. Furthermore, iPSCs generated from diseased cells can serve as useful tools for studying disease mechanisms and potential therapies. These induced pluripotent stem cells (iPSCs) offer a promising approach to patient-specific regenerative medicine therapies, and are attractive for studying mechanisms of disease and drug toxicity. To generate iPSCs from somatic cells, viral vectors or plasmids have been used to overexpress a combination of transactivating factors, e.g. Oct3/4, Sox2, c-Myc, Klf4, Lin28, and Nanog. However, these methods result in a low efficiency of reprogramming and fail to provide precise control of the reprogramming process. Furthermore, these methods for nuclear reprogramming inherently raise concerns about potential tumorigenicity and gene-silencing mutations caused by DNA integration.

The set of reprogramming factors (RFs) may be chosen from Oct3/4, Sox2, c-Myc, Klf4, Lin28, and Nanog. Oct3/4 and Sox2 are transcription factors that maintain pluripotency in embryonic stem (ES) cells while Klf4 and c-Myc are transcription factors thought to boost iPSC generation efficiency. The transcription factor c-Myc is believed to modify chromatin structure to allow Oct3/4 and Sox2 to more efficiently access genes necessary for reprogramming while Klf4 enhances the activation of certain genes by Oct3/4 and Sox2. Nanog, like Oct3/4 and Sox2, is a transcription factor that maintains pluripotency in ES cells while Lin28 is an mRNA-binding protein thought to influence the translation or stability of specific mRNAs during differentiation. Recently, it has been shown that retroviral expression of Oct3/4 and Sox2, together with co-administration of valproic acid, a chromatin destabilizer and histone deacetylase inhibitor, is sufficient to reprogram fibroblasts into iPSCs.

Though virally-generated iPSCs avoid ethical and immunogenicity issues surrounding embryonic stem cells, they are not fit for clinical use. DNA-based strategies to overexpress reprogramming factors are associated with exogenous DNA integration and may silence indispensable genes and/or induce tumorogenicity. Even with adenoviral and plasmid DNA induction of pluripotentiality, integration remains a concern. Moreover, the stochastic nature of nucleic acid-based infection and expression complicates characterization of the reprogramming process in terms of dosing and sequence of expression. Furthermore, the low efficiency of DNA-based strategies for nuclear reprogramming may be related in part to the inefficiencies of gene transfer. Thus, a non-viral and non-nucleic acid induction method may be preferred for safer and higher throughput generation of iPSCs for therapeutic applications.

Escherichia coli is a widely used organism for the expression of heterologous proteins. It easily grows to a high cell density on inexpensive substrates to provide excellent volumetric and economic productivities. Well established genetic techniques and various expression vectors further justify the use of Escherichia coli as a production host. However, a high rate of protein synthesis is necessary, but by no means sufficient, for the efficient production of active biomolecules. In order to be biologically active, the polypeptide chain has to fold into the correct native three-dimensional structure, and remain soluble at useful concentrations.

In many cases, the recombinant polypeptides have been found to be sequestered within large refractile aggregates known as inclusion bodies. Active proteins can be recovered from inclusion bodies through a cycle of denaturant-induced solubilization of the aggregates followed by removal of the denaturant under conditions that favor refolding. But although the formation of inclusion bodies can sometimes ease the purification of expressed proteins; in most occasions, refolding of the aggregated proteins remains a challenge.

For several decades, in vitro protein synthesis, also called cell-free protein synthesis (CFPS), has served as an effective tool for lab-scale expression of cloned or synthesized genetic materials. In recent years, in vitro protein synthesis has been considered as an alternative to conventional recombinant DNA technology, because of disadvantages associated with cellular expression. In vivo, proteins can be degraded or modified by several enzymes synthesized with the growth of the cell, and, after synthesis, may be modified by post-translational processing, such as glycosylation, deamidation or oxidation. In addition, many products inhibit metabolic processes and their synthesis must compete with other cellular processes required to reproduce the cell and to protect its genetic information.

Cell-free protein synthesis has the potential to replace bacterial fermentation as the technology of choice for the production of many recombinant proteins. The most significant advantage is that all of the resources in the reaction theoretically can be directed toward production of the desired product and not to secondary reactions, e.g., those that maintain the viability of the host cell. In addition, removing the need to maintain host cell viability allows the production of proteins that are toxic to the host cell. Furthermore, the lack of a cellular membrane allows direct access to the reaction volume, allowing for addition of reagents that increase the efficacy of the in vitro synthesis reaction (e.g., increase protein yield).

Improvements in in vitro synthesis systems that produce active mammalian proteins are of continued interest and are the subject of the present invention.

### SUMMARY OF THE INVENTION

Compositions and methods are provided for cell-free synthesis of reprogramming transcription factors (RF). The methods of synthesis allow the production of biologically active reprogramming factor compositions, of increased concentration, purity and solubility relative to conventional methods. As used herein, reprogramming factors are nuclear-acting polypeptides that alter transcription, which factors may induce pluripotency in targeted cells. Typically reprogramming factors of interest for the methods of the invention are fused to a polypeptide permeant domain, e.g. nona-arginine, tat, etc. as known in the art.

Improvements to the synthetic reaction include, without limitation, one or more of fusion of the reprogramming factor to a fusion partner that enhance solubility, and/or a partner that provides endosomolytic activity; which fusion partner is generally linked to the reprogramming factor through a define proteolytic cleavage site, e.g. a TEV protease cleavage site. Cleavage may be performed in a buffer optimized for maintaining solubility of the RF, e.g. buffer comprising from about 1 to about 3 M urea; buffer comprising suitable proteins and/or polynucleotides to maintain solubility, and the like.

The cell-free synthetic reactions may be altered from conventional methods in temperature, e.g. by decrease of temperature to not more than about 30° C., not more than about 25° C., and at least about 20° C., usually at least about 22° C. Usually the bacterial extracts provided in the reaction mixture for cell-free synthesis are extracts of bacteria that are genetically altered to have decreased endogenous protease activity. The extracts may have decreased concentrations of potassium glutamate relative to convention reactions, e.g. comprising about 15-25 mM magnesium glutamate in the absence of potassium glutamate.

Following synthesis, the solubility of the RF may be enhanced by on-column folding. In such a method, the polypeptide is solubilized in a high concentration of an agent such as urea. The solubilized polypeptide is bound to an affinity column through a suitable tag or epitope, e.g. biotin, HIS tag, etc., as known in the art. The bound polypeptide refolding by washing in decreasing concentrations of urea, then eluted from the column.

In cell free synthetic reactions, the modifications described herein provide for greater synthetic yield of soluble, biologically active protein, where biologically active protein may be measured by various methods, including PAGE, capillary electrophoresis, affinity analysis, functional analysis of protein activity, and the like, as known in the art. The use of the improvements provide at least a 20% improvement in the yield of the active protein as compared to conventional synthetic methods, and may provide for at least a 30%, at least a 40%, at least a 50%, at least a 75%, at least 100% or more improvement in yield.

### BRIEF DESCRIPTION OF THE DRAWINGS

FIG. 1. Modular Vector Design. (A) Schematic depiction of the R9 fusion protein design. CAT9: 5′ translation enhancer, consisting of the first nine amino acids of chloramphenicol acetyltransferase (CAT) sequence to destabilize mRNA secondary structure for more efficient initiation of translation (Son et al. 2006), STREP: Strep Tag II purification tag, PCS: Protease cleavage site, Factor Xa was chosen for this construct to enable removal of the translation enhancer, if deemed necessary. R9: Nona-arginine translocation signal, HIS: Hexa-histidine purification tag, LNK: Linker sequence, GGGGS, to physically separate the N-terminal R9 fusion peptide from the RF cargo. Reprogramming Factor Coding Sequence: coding sequence for human transcription factors Oct3/4, Sox2, c-Myc, Klf4, Lin28, and Nanog. (B) DNA and amino acid sequences for the N-terminal R9 fusion peptide on the modular plasmid. 150×64 mm (300×300 DPI).

FIG. 2. Autoradiography analysis of fusion reprogramming factor proteolysis. 4-μL samples of soluble CFPS product were separated by SDS-PAGE. (A) Analysis of R9-Nanog proteolysis. 1: KC6 extract, expressed at 37° C. 2: KC6 extract, 25° C. 3: KC6+Roche protease inhibitor, 25° C. 4: BL21(DE3)Star protease-deleted extract, 25° C. (B) Insight gained from solving R9-Nanog proteolysis is transferable to R9-Sox2. 5: KC6 extract, 37° C. 6: BL21(DE3)Star protease-deleted extract, 25° C. Representative images of individual lanes were selected from experiments performed on different days. 80×43 mm (300×300 DPI).

FIG. 3. Autoradiography and scintillation counting analysis of temperature-dependent effects on soluble protein production. 4-μL samples of total (T) and soluble (S) CFPS final reaction mixture product are separated by SDS-PAGE. Lowering R9-Nanog production temperature from 37° C. to 25° C. resulted in a slight improvement in soluble protein yield. This insight was also applied to R9-Oct3/4 and resulted in a significant improvement in soluble protein yield. Autoradiograms and scintillation counting data were selected from representative experiments performed on different days. 80×73 mm (300×300 DPI).

FIG. 4. Autoradiography for Yamanaka and Thomson reprogramming factor R9 fusion proteins produced using CFPS. 4-μL samples of total (T) and soluble (S) CFPS reaction products were separated by SDS-PAGE. All transcription factors are nona-arginine fusion proteins as described in FIG. 1. The top row shows initial results obtained with KC6 extract and 37° C. production temperature. The same panel of reprogramming factors was produced with BL21(DE3)Star cell extract at 25° C. to generate improve yields of soluble and full-length proteins as shown in the bottom row. 80×51 mm (300×300 DPI).

FIG. 5. Scalable cell-free production of soluble R9-Nanog, R9-Sox2, and R9-Oct3/4 for characterization studies. The following are taken from representative reactions that incorporate the optimized production parameters presented above. Multiple 1-mL reactions were co-processed up to 80-mL total volumes to obtain greater amounts of fusion protein for characterization studies. 80×46 mm (300×300 DPI).

FIG. 6. Competitive analysis (NoShift assay) of R9-Nanog (A), R9-Oct3/4 (B), R9-Sox2 (C) showing that nona-arginine fusion proteins retain their DNA binding activity. When incubated with Biotinylated cognate consensus sequence, R9-fusion protein-DNA binding was observed. Specific competitor DNA with non-biotinylated cognate consensus sequence significantly reduced the binding activity in each R9-fusion protein, confirming sequence-specificity of the assay for R9-fusion protein binding. Non-biotinylated scrambled nonsense sequence had no effect on R9-fusion protein binding. As a positive control, human recombinant proteins (rhNanog and rhSox2) were used. 99×118 mm (300×300 DPI).

FIG. 7. R9-Nanog translocation in mouse embryonic fibroblasts. Cells were treated with 0.5 μM R9-Nanog for 2 hrs at 37° C. Red indicates Nanog by Alexa-Fluor 594 labeled antibody detection and blue indicates nuclei by DAPI stain. (A) Fluorescence microscopy of R9-Nanog-treated cells. (B) Fluorescence microscopy of commercial Nanog-treated cells. Fluorescence Microscopy; 40× magnification. (C) Confocal microscopy of R9-Nanog-treated cells. (D) Confocal microscopy of commercial Nanog-treated cells. Confocal Microscopy; 63× magnification. 99×74 mm (300×300 DPI).

FIG. 8. CFPS-produced R9-50×2 induces downstream target gene expression while rSOX2 does not. Control=negative control, no transcription factor is added to the cell culture media. rSOX2 was also produced by CFPS but lacks the R9 transduction domain. Intracellular SOX2 expression after recombinant retroviral infection was used as a positive control (solid horizontal line). 100 nM of the respective protein was added every 24 h for 4 days and gene expression levels were determined for cell samples taken at each time point.

FIG. 9: Multiple, simultaneous CFPS reactions were used to conveniently compare multiple reaction conditions in which the environment for polypeptide elongation and folding can be precisely modified to encourage optimal protein expression and folding.

### DETAILED DESCRIPTION OF THE EMBODIMENTS

Compositions and methods are provided for the cell-free synthesis of biologically active reprogramming transcription factors, providing improvements for enhancing the solubility and biological activity of the RF. These methods are applicable to continuous, semi-continuous and batch reactions.

Cell-free protein synthesis (CFPS) technology can potentially be applied to facilitate the production of recombinant RFs for a non-viral approach. Briefly, E. coli are lysed at high pressures to extract both protein production machinery as well as inner membrane vesicles for energy regeneration. This extract is incubated with template DNA encoding the protein of interest and a chemical environment that mimics the E. coli cytoplasm to yield the protein of interest (Jewett and Swartz 2004). Decoupling protein production from maintenance of host cell health permits production of toxic proteins and also concentrates energy and resources toward synthesis of the protein of interest (Wuu and Swartz 2008). In addition, the cell-free platform is an established system for efficiently producing effective therapeutic fusion protein cancer vaccines (Kanter et al. 2007). Thus, CFPS can potentially address toxicity and aggregation, which are two main roadblocks in fusion protein expression. The open nature of CFPS also enables easy and high-throughput manipulation of protein production parameters for optimizing protein expression conditions.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

Reprogramming factors, as used herein, refers to one or a cocktail of biologically active polypeptides that act on a cell to alter transcription, and which upon expression, reprogram a cell to multipotency or to pluripotency. For the purposes of the present invention, reprogramming factors are usually fused to a permeant domain to allow entry of the polypeptide across a cell membrane and across the nuclear membrane. Reprogramming factors may be of any suitable mammalian species, e.g. human, murine, porcine, equine, canine, ovine, feline, simian, etc. Human polypeptides are of particular interest.

In some embodiments the reprogramming factor is a transcription factor, including without limitation, Oct3/4; Sox2; Klf4; c-Myc; and Nanog. Also of interest as a reprogramming factor is Lin28, which is an mRNA-binding protein thought to influence the translation or stability of specific mRNAs during differentiation.

The reprogramming factors may be provided as a composition of isolated polypeptide, i.e. in a cell-free form, which is biologically active. Biological activity may be determined by specific DNA binding assays, as described in the Examples; or by determining the effectiveness of the factor in altering cellular transcription. A composition of the invention may provide one or more biologically active reprogramming factors. The composition may comprise at least about 50 μg/ml soluble reprogramming factor, at least about 100 μg/ml; at least about 150 μg/ml, at least about 200 μg/ml, at least about 250 μg/ml, at least about 300 μg/ml, or more.

Examples of RF polypeptides include, but are not limited to, molecules such as derivatives and fragments of any of the above-listed polypeptides.

Permeant Domain. A number of permeant domains are known in the art and may be used in the present invention, including peptides, peptidomimetics, and non-peptide carriers. In one embodiment, the permeant peptide is derived from the third alpha helix of Drosophila melanogaster transcription factor Antennapaedia, referred to as penetratin, which comprises the amino acid sequence RQIKIWFQNRRMKWKK. In another embodiment, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

Solubility domain. The RF is optionally fused to a polypeptide domain that increases solubility of the product. Usually the domain is linked to the RF through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase RF solubility, and the like.

Domains of interest include endosomolytic domains, e.g. influenza HA domain; and other polypeptides that aid in production, e.g. IF2 domain, GST domain, GRPE domain, and the like.

Cell-free synthesis. The RF protein is produced by cell-free synthesis, in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. A number of reaction chemistries for polypeptide synthesis can be used in the methods of the invention. For example, reaction chemistries are described in U.S. Pat. No. 6,337,191, issued Jan. 8, 2002, and U.S. Pat. No. 6,168,931, issued Jan. 2, 2001, herein incorporated by reference.

In one embodiment of the invention, the reaction chemistry is as described in international patent application WO 2004/016778, herein incorporated by reference. The activation of the respiratory chain and oxidative phosphorylation is evidenced by an increase of polypeptide synthesis in the presence of O₂. In reactions where oxidative phosphorylation is activated, the overall polypeptide synthesis in presence of O₂ is reduced by at least about 40% in the presence of a specific electron transport chain inhibitor, such as HQNO, or in the absence of O₂. Improved yield is obtained by a combination of factors, including the use of biological extracts derived from bacteria grown on a glucose containing medium; an absence of polyethylene glycol; and optimized magnesium concentration. This provides for a homeostatic system, in which synthesis can occur even in the absence of secondary energy sources.

The template for cell-free protein synthesis can be either mRNA or DNA. Translation of stabilized mRNA or combined transcription and translation converts stored information into protein. The combined system, generally utilized with a bacterial extract, e.g. an Enterobacteriaceae extract, including E. coli, Erwinia, Pseudomonas, Salmonella, etc., continuously generates mRNA from a DNA template with a recognizable promoter. Either endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Ammonium may be added at from between 0-100 mM. The pH of the reaction is generally between pH 6 and pH 9. The temperature of the reaction is generally between 20° C. and 40° C., where lower temperatures are preferred, e.g. around about 20° C., around about 25° C., around about 30° C. These ranges may be extended.

Metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

Vesicles, either purified from the host organism or synthetic, may also be added to the system. These may be used to enhance protein synthesis and folding. This cytomim technology has been shown to activate processes that utilize membrane vesicles containing respiratory chain components for the activation of oxidative phosphorylation.

Synthetic systems of interest include the transcription of RNA from DNA or RNA templates, and the translation of RNA into polypeptides.

The reactions may be large scale, small scale, or may be multiplexed to perform a plurality of simultaneous syntheses. Additional reagents may be introduced to prolong the period of time for active synthesis. Synthesized product is usually accumulated in the reactor, and then is isolated and purified according to the usual methods for protein purification after completion of the system operation.

Of particular interest is the translation of mRNA to produce proteins, which translation may be coupled to in vitro synthesis of mRNA from a DNA template. Such a cell-free system will contain all factors required for the translation of mRNA, for example ribosomes, amino acids, tRNAs, aminoacyl synthetases, elongation factors and initiation factors. Cell-free systems known in the art include E. coli extracts, etc., which can be prepared using a variety of methods. Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki et al. (1992) Anal Biochem 206(2):389-93 modify the S30 E. coli cell-free extract by collecting the ribosome fraction from the S30 by ultracentrifugation. Zawada and Swartz Biotechnol Bioeng, 2006. 94(4): p. 618-24 teach a modified procedure for extract preparation.

In addition to the above components such as cell-free extract, genetic template, and amino acids, materials specifically required for protein synthesis may be added to the reaction. These materials include salts, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, etc.

The salts preferably include magnesium, and ammonium salts of acetic acid or glutamic acid, and some of these may have an alternative amino acid as a counter anion. The polymeric compounds may be polyethylene glycol, dextran, diethyl aminoethyl dextran, quaternary aminoethyl and aminoethyl dextran, etc. The oxidation/reduction adjuster may be dithiothreitol, ascorbic acid, cysteine, glutathione and/or their oxides. Also, a non-denaturing surfactant such as Brij-35 may be used at a concentration of 0-0.5 M. Spermine and spermidine may be used for improving protein synthetic ability, and cAMP may be used as a gene expression regulator.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

The amount of protein produced in a translation reaction can be measured in various fashions. One method relies on the availability of an assay which measures the activity of the particular protein being translated. Examples of assays for measuring protein activity are a DNA binding assay system. These assays measure the amount of functionally active protein produced from the translation reaction. Activity assays will not measure full-length protein that is inactive due to improper protein folding or lack of other post translational modifications necessary for protein activity.

Another method of measuring the amount of protein produced in a combined in vitro transcription and translation reactions is to perform the reactions using a known quantity of radiolabeled amino acid such as ³⁵S-methionine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

In some embodiments, the synthetic reactions are performed in the substantial absence of polyethylene glycol (PEG), e.g. PEG at a concentration of less than about 0.1%, and may be less than about 0.01%. Spermine or spermidine is then present at a concentration of at least about 0.5 mM, usually at least about 1 mM, preferably about 1.5 mM, and not more than about 5 mM. Putrescine is present at a concentration of at least about 0.5 mM, preferably at least about 1 mM, preferably about 1.5 mM, and not more than about 5 mM. The reaction mix may comprise less than about 1 mM potassium glutamate and may be substantially free of potassium glutamate, and may comprise magnesium glutamate at a concentration of from about 1 mM, 5 mM, 10 mM, 20 mM, and not more than about 30 mM.

Following synthesis, the solubility of the RF may be enhanced by on-column folding. In such a method, the polypeptide is solubilized in a high concentration of an agent such as urea. The solubilized polypeptide is bound to an affinity column through a suitable tag or epitope, e.g. biotin, HIS tag, etc., as known in the art. The bound polypeptide refolding by washing in decreasing concentrations of urea, then eluted from the column.

Biological extracts. For the purposes of this invention, biological extracts are any preparation comprising the components of protein synthesis machinery, usually a bacterial cell extract, wherein such components are capable of translating a nucleic acid encoding a desired protein. Thus, a bacterial extract comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N10-formyltetrahydrofolate, formylmethionine-tRNAfMet synthetase, peptidyl transferase, initiation factors such as IF-1, IF-2 and IF-3, elongation factors such as EF-Tu, EF-Ts, and EF-G, release factors such as RF-1, RF-2, and RF-3, and the like.

In some embodiments, the extract is prepared from a bacterial strain that is deficient in proteases, e.g. one or more of OmpT and Lon proteases. For example, OmpT and/or Lon proteases may be inactivated by deletion, insertion of stop codons, etc. For convenience, the organism used as a source of extracts may be referred to as the source organism. In certain embodiments of the invention, the reaction mixture comprises extracts from bacterial cells, e.g. E. coli S30 extracts, as is known in the art. Many different types of bacterial cells have been used for these purposes, e.g. Pseudomonas sp., Staphylococcus sp., Methanococcus sp., Methanobacterium sp., Methanosarcina sp., etc. In certain of these embodiments, the bacterial cell contains a deletion or directed mutation of a specific gene. Specific genetic modifications of interest include modifications to the proteases

Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki et al. (1992) Anal Biochem 206(2):389-93 modify the S30 E. coli cell-free extract by collecting the ribosome fraction from the S30 by ultracentrifugation. While such extracts are a useful source of ribosomes and other factors necessary for protein synthesis, they can also contain small amounts of enzymes responsible for undesirable side-reactions that are unrelated to protein synthesis, but which modulate the oxidizing environment of the reaction, and which can act to reduce the groups on the nascent polypeptide and the redox buffer.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

In this work, we used an E. coli based cell-free protein synthesis (CFPS) platform to express the above set of six RFs as fusion proteins, each with a nona-arginine protein transduction domain. Using the flexibility offered by the CFPS platform, we successfully addressed proteolysis and protein solubility problems to produce full-length and soluble R9-RF fusions. We subsequently showed that R9-Nanog, R9-Oct3/4, and R9-Sox2 retain their DNA-binding function, and that the R9-RF construct is capable of translocating across the plasma and nuclear membranes of mouse embryonic fibroblasts. R9-RF fusion proteins produced using the CFPS platform were full-length, soluble, transducible, and retained their DNA-binding activities. These methods allow the realization of a non-viral fusion protein approach for iPSC generation.

### Materials and Methods

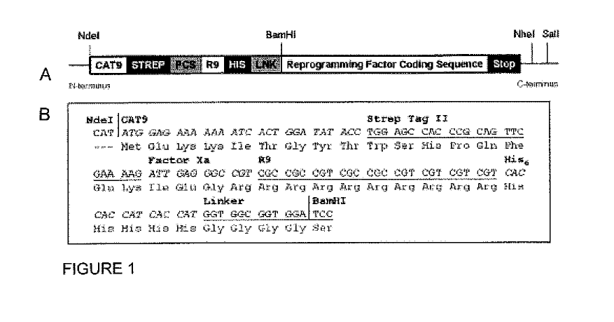

Plasmid Construction. Genes for six human RFs as well as the DNA sequence encoding the N-terminal R9 fusion peptide (FIG. 1) were codon-optimized for expression in E. coli using DNAworks (Hoover and Lubkowski 2002). In addition, formation of stable mRNA secondary structures near the start codon was minimized using MFOLD (Zuker 2003). MFOLD was used to choose the first nine codons encoding the chloramphenicol acetyltransferase translation enhancer placed at the 5′ end of the coding sequence. Overlapping oligonucleotides were designed for PCR based gene synthesis using DNAworks. A two-step gene synthesis PCR method was utilized to generate RF coding sequences (Reisinger et al. 2006). The N-terminal R9 fusion peptide sequence (FIG. 1) was synthesized as part of the Nanog gene, with a 5′ NdeI restriction site containing the start codon and 3′ NheI and SalI sites following the stop codon. The remaining genes were synthesized with 5′ BamHI sites near the start codon and 3′ NheI sites following the stop codon.

PCR products were first cloned into pCR2.1TOPO (Invitrogen) following the manufacturer's instructions. The R9-Nanog fusion gene was then cloned into a pET24a (Novagen) expression vector between the T7 promoter and terminator using NdeI and SalI restriction sites. The modular design of the R9-RF fusions enabled facile assembly of the other expression plasmids by replacing the Nanog gene in pET-24a-R9-Nanog with the other RF coding sequences using BamHI and NheI restriction sites. The resulting pET24a-based expression vectors for R9-RF fusions were verified by DNA sequencing (Table 1). Milligram quantities of plasmid were isolated from E. coli cultures grown in Terrific Broth (Invitrogen) using Maxiprep and Gigaprep kits (Qiagen) according to the manufacturer's instructions.

E. coli Cell Extract Preparation. E. coli KC6 (Calhoun and Swartz 2006), and BL21(DE3)Star (Invitrogen) cell extracts were prepared from cells grown in one of two formats: (1) small-scale shake flasks and (2) 10-liter high-density fermentations. In the shake flask format, cells were grown in 0.5 L of defined media (Zawada and Swartz 2005) or 2YT media in 2 L shake flasks and harvested during midlate logarithmic growth at 3 to 6 OD600. In the high-density fermentation format, cells were grown in a B. Braun C-10-2 No. 153 15-L fermentor on defined media with glucose and amino acid feeds using a procedure that promotes logarithmic growth to moderate cell density while avoiding acetate accumulation (Zawada and Swartz 2005). The fermentation was harvested at approximately 30 OD₆₀₀. Cell growth conditions (shake flask v. high-density, temperatures ranging from 20° C. to 37° C., defined media v. 2YT) were observed to produce comparable extracts in terms of protein productivity and protease activities at a cell-free expression temperature of 25° C. In BL21 cultures, 1 mM IPTG was added at 0.6 OD₆₀₀ to induce T7 RNA polymerase (RNAP) expression for CFPS.

Immediately after the growth/expression period, cultures were immediately centrifuged at 5000-8000 g for 30 min at 4° C. and washed at 4° C. by resuspending in cold S30 buffer (10 mM Tris-acetate pH 8.2, 14 mM magnesium acetate, and 60 mM potassium acetate) and recentrifuged. The centrifugation and wash procedure was repeated one to two additional times, and the resulting cell paste was stored at −80° C. until it was processed into S30 cell extract. Frozen cell paste was thawed in 0.8 mL of S30 buffer per 1 gram of cell paste and suspended to homogeneity with a Model 700 rotary homogenizer (Fisher Scientific). The cells in suspension were lysed by a single pass through an Emulsiflex C-50 (Avestin) high-pressure homogenizer at 17,500 to 25,000 psi. The homogenate was clarified by centrifugation at 30,000×g at 4° C., twice for 30 min each, and the resulting pellets were discarded. The supernatant was then incubated for 80 minutes at 37° C. in the dark on a rotary shaker at 120 rpm. After this incubation, the cell extract was flash-frozen and stored at −80° C.

Cell-free Production of Fusion Reprogramming Factors. CFPS was conducted using the PANOx-SP (PEP, Amino Acids, nicotinamide adenine dinucleotide (NAD), Oxalic Acid, Spermidine, and Putrescine) system as described previously with minor changes in component concentrations (Jewett and Swartz 2004). The modifications were: 20 mM magnesium glutamate, 0.17 mg/mL folinic acid, 85.3 μg/mL E. coli tRNA mixture (Roche Molecular Biochemicals), 2.7 mM potassium oxalate. Reagents were obtained from Sigma-Aldrich unless otherwise noted. For protease inhibitor studies, one Protease Inhibitor Cocktail tablet (Roche Molecular Biochemicals) was dissolved in 500-μL of sterile water. Protease inhibitor solution was added in place of water typically used to bring CFPS reactions up to volume. CFPS reactions were conducted at 20-μL volumes in 1.5-mL Eppendorf tubes for small-scale diagnostic purposes and at 1-mL volumes in 6-well tissue culture plates (BD Falcon) for preparative purposes. Reactions were carried out at 37° C., 30° C. or 25° C. for 3 hours.

Quantification of protein yields by liquid scintillation counting. Following the cell-free reaction period, samples were placed on ice to stop the reaction. 3-5 μL of cold reaction mixture was spotted on filter paper and allowed to dry. The remainder of the CFPS reaction was centrifuged at 20,800×g for 15 minutes at 4° C. to isolate the soluble fraction of the protein product. An equal volume of the supernatant was spotted on filter paper and allowed to dry. Total and soluble protein concentrations were then estimated using the trichloroacetic acid procedure described previously to precipitate the synthesized protein (Calhoun and Swartz 2005). The L-[U-14C]-Leucine radioactivity was quantified by a LS3801 liquid scintillation counter (Beckman Coulter). Total and soluble protein yields were calculated based on incorporated radioactivity and the leucine content of the protein of interest.

Assessment of Proteolysis and Solubility by SDS-PAGE and Autoradiography. 3-5 μL of total and soluble protein fractions were loaded onto NuPAGE 10% Bis-Tris gels (Invitrogen) for protein quantification and characterization. Samples were run in MOPS running buffer (Invitrogen) under non-reducing conditions as RFs are non-disulfide bonded. Gels were stained, dried, and exposed to a storage phosphor screen (Molecular Dynamics) which was subsequently scanned using a Typhoon Scanner (GE Healthcare).

Ni-NTA Affinity Chromatography of Fusion Reprogramming Factors for Characterization. Following preparative-scale CFPS reactions, the soluble protein fraction was obtained after centrifugation at 20,800×g for 15 minutes at 4° C. The supernatant was dialyzed against 100 volumes of loading buffer (LB, 300 mM NaCl, 10 mM imidazole, 50 mM phosphate buffer, pH 8.0) with 2 buffer changes for 3 hours each at 4° C. and loaded on a 1-mL Ni-NTA column equilibrated with wash buffer (WB, 30 mM imidazole in LB). The column was washed with 6 column volumes (CV) of WB and eluted with increasing imidazole concentrations (1 CV each of 100, 175 and 250 mM imidazole in WB). After pooling appropriate elution fractions, eluate was simultaneously concentrated and buffer-exchanged into a 20% sucrose-PBS formulation using a 10-kDa molecular weight cutoff Amicon Ultra-4 centrifugal device. Protein concentrations were quantified using a DC Protein Assay (BioRad). Protein solutions were flash-frozen in liquid nitrogen and stored at −80° C. until characterization or use.

Assessment of the DNA Binding Activities of the Fusion Reprogramming Factors. The DNA-binding activities of the R9-Nanog, R9-Oct3/4, and R9-Sox2 fusion proteins were assayed by colorimetry utilizing the NoShift Transcription Factor Assay Kit (Novagen) according to the manufacturer's instructions. To assess sequence-specific binding activity, 1-2 μg of the R9-Nanog fusion protein, 2-10 μg of the R9-Oct3/4 fusion protein or 1-4 μg of the R9-Sox2 fusion protein were each incubated in 204 with 0.5 μM of their respective biotinylated consensus dsDNA binding targets. (Oligos for Nanog cognate consensus sequence: ACC TGT TAA TGG GAG CGC; Oct3/4 consensus sequence: GCA GAG AGA TGC ATG TGC CGT; Sox2 consensus sequence: GCA GAG GAC AAA GGT GCC GTG). Non-biotinylated competitor consensus dsDNA and scrambled dsDNA used to assess competitive binding were added at 2.5 W. As a positive control, recombinant human (rh) Nanog (Abcam) and rhSox (Peprotech) were used. HRP-conjugated anti-mouse immunoglobulin G was used as a secondary antibody to recognize the anti-Nanog (Abcam), anti-Oct3/4 (R&D Systems), and anti-Sox2 (R&D Systems) mouse monoclonal antibodies. All assays were performed in duplicate. Binding activity was measured via colorimetric absorbance at 450 nm on a Tecan multiwell spectrophotometer using 3,3,5,5-tetramethylbenzidine as the substrate.

Assessment of the Cell Transducibility of the Fusion Reprogramming Factors. Mouse embryonic fibroblasts (MEFs) were seeded on gelatin-coated coverslips (VWR micro cover glass) and grown in fully supplemented Dulbecco's Modified Eagle Medium (DMEM; GIBCO) overnight at 37° C. in a 5% CO₂ incubator. MEFs have a large cytoplasm-to nucleus ratio that allow for better visualization of protein localization. After 24 h, media was aspirated and cells were washed with phosphate buffered saline (PBS; GIBCO) twice. The R9-Nanog fusion protein was diluted to a final concentration of 500 nM with serum-free DMEM and added to the cells. Control groups were either treated with equimolar concentrations rhNanog (Abcam) diluted in serum-free DMEM or simply with serum-free DMEM of equal volume. Cells were incubated at 37° C. for 2 hours to allow protein uptake (translocation) and then fixed for immunostaining using 4% paraformaldehyde (Alfa Aesar) for 10 min at room temperature (RT). Subsequently, the cells were washed with ice-cold PBS twice and permeabilized with 0.25% Triton-X (in PBS) for 10 min at RT. The permeabilized cells were then washed in PBS three times and blocked with a 2% non-fat milk and 10% normal goat serum (Chemicon) mixture for 1 hr at RT. Finally, cells were incubated overnight at 4° C. with mouse anti-human Nanog monoclonal antibody (Abcam; ab62734) diluted (1:250) in blocking solution. In parallel, negative assay controls with no primary antibodies were included. To remove unbound primary antibody, the cells were washed in PBS three times and incubated with Alexa-Fluor 594 conjugated goat anti-mouse (1:200) secondary antibody (Molecular Probes; A11032) for 1 hr at RT. Excess antibody was washed off and slides were mounted with mounting media containing DAPI (Santa Cruz Biotechnology; sc-24941) and examined by both fluorescence (Nikon Eclipse TE2000-U) and confocal (Zeiss LSM 510 Dual Photon) microscopy.

### Results

Construction of the Fusion Reprogramming Factors. Successful generation of iPSCs requires both intracellular as well as intranuclear delivery of RFs. The six RFs examined here are all transcription factors possessing native nuclear localization sequences (NLS) for nuclear entry. But, they do not contain a sequence for cell entry. Our modular pET24a-based R9-RF expression vector confers cell transducibility onto each sub-cloned entity (FIG. 1). The R9 sequence is believed to bind to heparan sulfate on the cell surface, thereby triggering cell uptake (Fuchs and Raines 2004). Fusion RFs were initially screened for expression at the 20-μL diagnostic reaction scale using the PanOx-SP CFPS system with E. coli cell extract derived from KC6, an amino acid-stabilized A19 strain, at the standard protein production temperature of 37° C. Scintillation counting and autoradiograms of CFPS produced proteins analyzed by SDS-PAGE indicated the accumulation of mostly truncated and insoluble products below the expected molecular weight. Since R9-Nanog exhibited both truncation and solubility problems, it was selected as the model protein for troubleshooting.

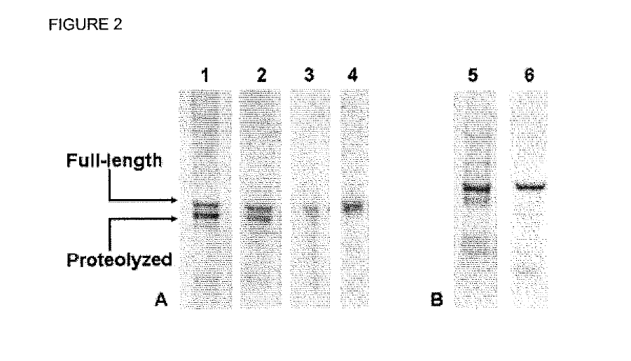

Identification and Abrogation of Proteolysis. Production of R9-Nanog using standard conditions yielded two polypeptide populations: one with the expected molecular weight and one with a prominent band approximately 2 kDa below the expected molecular weight (FIG. 2A). Lowering the production temperature from 37° C. to 25° C. reduced the intensity of the truncated band, but it was still unclear whether truncation was due to (1) incomplete translation or (2) proteolysis. Suspecting that the R9 sequence offered a vulnerable protease target, we took advantage of the open cell-free system and supplemented the 25° C. reaction with Roche Protease Inhibitor Cocktail. A resulting single band corresponding to the full-length R9-Nanog fusion protein appeared on the autoradiogram, thereby implicating proteolysis. Though protease inhibitor was an effective diagnostic tool, it was not a practical solution. Not only did it reduce total protein yield, but it also added a significant expense, especially for downstream processing since its removal by dialysis allowed proteases to regain activity. Thus, bacterial cell extract was prepared from E. coli BL21(DE3)Star, a commercially-available strain deficient in OmpT and Lon proteases. By substituting the standard extract with the protease-deficient extract, we significantly reduced proteolysis without the use of protease inhibitors (FIG. 2).

We were able to apply conditions for producing full-length R9-Nanog to successfully produce other proteolysis-prone R9-RF fusion proteins. R9-Sox2 produced under standard conditions also exhibited lower molecular weight products (FIG. 2B). Use of BL21(DE3)Star extract at 25° C. also effectively curbed R9-Sox2 proteolysis.

Improving Protein Solubility. With proteolysis resolved, we moved on to solubility. Solubility is a problem that all six RFs share. Thus, we evaluated a variety of production schemes for enhancing solubility such as (1) supplementing CFPS reactions with molecular chaperones, (2) supplying the CFPS reaction with the cognate consensus dsDNA of respective RFs, and (3) lowering production temperature. Often, aggregation of incorrectly-folded polypeptides is the root cause of poor solubility. Overexpression of molecular chaperones has been reported to improve soluble yields (de Marco 2007), but supplementing our RF CPFS reactions with chaperones such as BiP, DnaK, and GroES/GroEL did not improve solubility (data not shown). Further, many proteins undergo conformational changes upon binding their consensus dsDNA (Spolar and Record 1994).

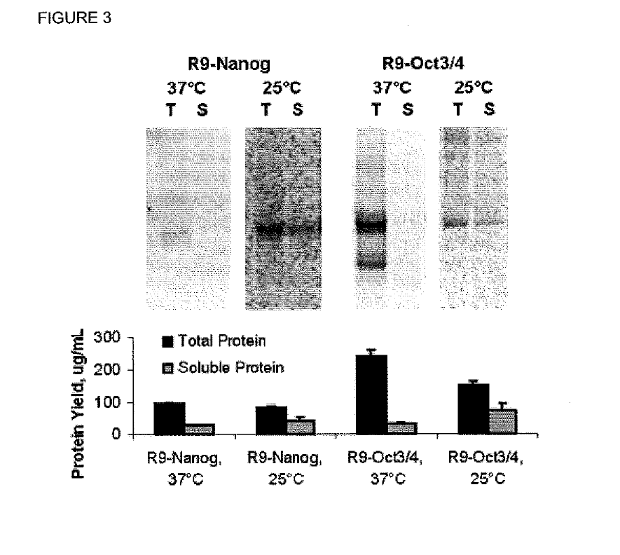

However, adding consensus dsDNA to respective R9-Nanog, R9-Oct3/4, and R9-Sox2 CFPS reactions also had no effect on solubility. Of the three remedies tested, only lowering production temperature yielded modest improvements in solubility. Production of soluble R9-Nanog was screened at 37° C., 30° C., and 25° C. The 25° C. production temperature yielded the best results for R9-Nanog (FIG. 3). Again, we sought to apply this solubility improving condition to other proteins experiencing similar problems with solubility. R9-Oct3/4 was one of the most recalcitrant proteins in terms of solubility; a major portion of the synthesized product formed insoluble aggregates. As with R9-Nanog, lowering production temperature yielded a nearly two-fold improvement in the accumulation of soluble R9-Oct3/4 (FIG. 3).

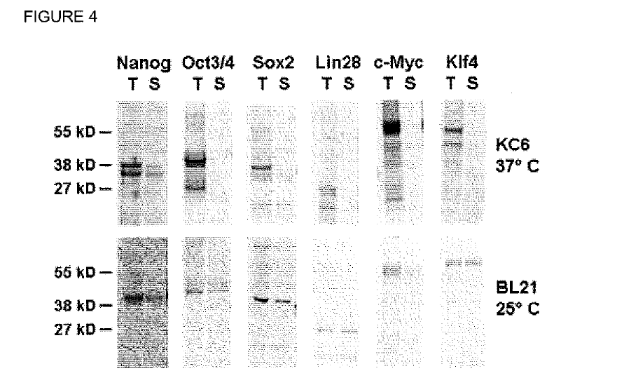

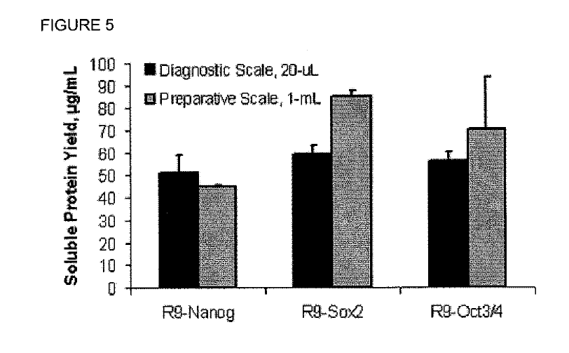

Generation of Full-length and Soluble Fusion Reprogramming Factors. The proteolysis and solubility studies on R9-Nanog yielded an optimized set of production conditions for the synthesis of full-length and soluble fusion RFs. We applied the optimized conditions to our set of six fusion RFs and were able to curb proteolysis and improve solubility for each RF (FIG. 4). While total protein yields range from 100-200 μg/mL, percent solubilities range from 20-40%. Thus, solubility problems still persist. While the fusion RFs are full-length and soluble, we must verify that (1) the N-terminal R9 fusion peptide does not interfere with the RFs' DNA binding function and (2) that R9 confers cell transducibility. In order to produce sufficient amounts of protein for characterization studies, we scaled up the production of R9-Nanog, R9-Sox2, and R9-Oct3/4 using the optimized conditions described above. Scintillation counting of the product showed comparable soluble protein yields from 20-μL and 1-mL reactions (FIG. 5). In order to produce sufficient amounts of protein, multiple 1-mL reactions could be easily co-processed. In this work, 20-mL batches were processed. Following protein synthesis, reaction mixtures were first centrifuged and product was isolated using a Ni-NTA column. Eluted protein samples were dialyzed into 20% sucrose-PBS, flash frozen, and stored at −80° C. before further characterization.

Fusion Reprogramming Factors Retain DNA Binding Activity. The NoShift Assay (Novagen) was used to verify the DNA binding activities of R9-Nanog, R9-Sox2, and R9-Oct3/4. The No-Shift Assay is a plate-based alternative to the electrophoretic mobility shift assay (EMSA), and is based on the same principles as EMSA. Briefly, fusion RFs and commercial recombinant protein positive controls were each incubated with their corresponding biotinylated cognate consensus dsDNA binding sequences. Protein-DNA complexes were then bound on a streptavidin-coated 96-well plate, and unbound complexes were washed away. Monoclonal primary antibodies specific for the RFs and fluorescently-labeled polyclonal secondary antibodies were used to probe for the bound complexes.

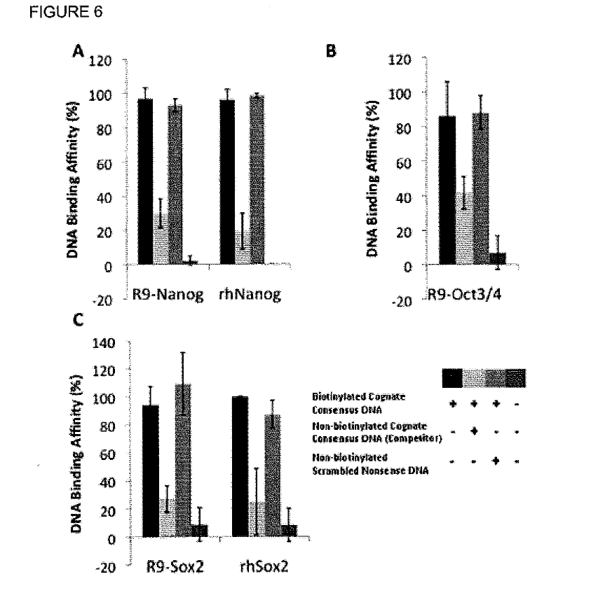

Fusion RFs behave similarly to their corresponding commercial recombinant proteins (FIG. 6). The cell-free extract, which served as a negative control, did not yield a signal. Co-incubation of the biotinylated consensus DNA with non-biotinylated consensus DNA sequences lowered the binding signal, which suggest that binding was specific and competitive. The binding of R9-RFs to their consensus sequences was comparable to that of the commercially available transcription factors. Further, co-incubation of the biotinylated consensus DNA with non-biotinylated scrambled nonsense DNA did not diminish the level of the protein-DNA complex. These results show that the R9-RFs retain DNA binding specificity.

R9 Fusion Construct Successfully Translocates across the Plasma Membrane. Cellular translocation studies were performed to verify that the N-terminal R9 confers cell transducibility. R9-Nanog was chosen to demonstrate the cell-transducing ability of our R9 fusion construct. Briefly, R9-Nanog was incubated with MEFs for 2 h after which noninternalized R9-Nanog was washed away. Cells were fixed and stained with primary antibodies specific for Nanog and fluorescently-labeled secondary antibodies. Internalization was visualized using fluorescence and confocal microscopy.

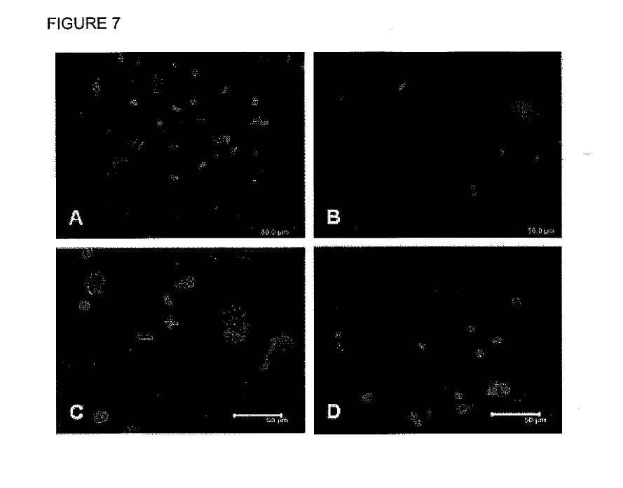

Fluorescence and confocal microscopy analyses suggest that the R9-Nanog effectively enters the cells (FIG. 7). Neither commercial recombinant Nanog nor the DMEM-treated control cells showed staining in any cell compartments. In the time course used in these studies, the R9-Nanog signal was predominantly observed as granular structures in a perinuclear location.

CFPS enables the production of appreciable amounts of full-length, soluble, and transducible nona-arginine fusion RFs that retain DNA-binding activity. Proteolysis was curtailed while solubility was enhanced. Lowering temperature reduced proteolysis and also improved product solubility. Transcription and translation rates are slower at lower temperatures, providing the growing polypeptide more time to explore the protein folding landscape and find its correct conformation.

Though our results are encouraging, there is room for improvement. Enhancing solubility is a key priority as we are currently losing 60-80% of total proteins produced to insoluble aggregates. Thus, protein refolding studies are underway in order to recover functional R9-RF fusion proteins from insoluble aggregates. We hope to gain insights into measures that will improve proper folding, which can be transferred into our cell-free production environment. Despite these solubility limitations, cell-free technology has enabled us to obtain appreciable amounts of protein for characterization studies. DNA-binding assays show that R9-Nanog, R9-Oct3/4, and R9-Sox2 are indeed correctly folded and are expected to serve as active transcription factors. The non-biotinylated consensus DNA and the scrambled DNA show the proteins' specificity for their respective binding partners. The data also show that the N terminal R9 fusion peptide that confers transducibility does not affect DNA-binding.

Successful translocation of R9-Nanog into MEFs completes the picture. Confocal microscopy shows that R9-Nanog crosses the plasma membrane. Intracellular delivery of R9-Nanog implies that the R9 internalization tag is accessible on the protein construct and has served its purpose. The perinuclear localization of R9-Nanog is in agreement with the PTD literature. It is hypothesized that the positively-charged R9 and other PTDs bind nonspecifically with heparan sulfate on cell surfaces. This binding event triggers macropinocytosis of the PTD fusions, thereby placing the internalized PTD fusions into endosomal vesicles.

The granular appearance of our R9-Nanog signal suggests that the fusion protein follows the proposed endosomal sequestration model. Nevertheless, preliminary functional studies in our laboratories indicate that a small fraction of the sequestered protein escapes the endosomes, and alters gene transcription.

In order to enhance our non-viral fusion protein approach for generating iPSCs, methods to increase endosomal escape are under investigation. Endosomal escape is possible with the addition of endosomolytic chemicals (Shiraishi et al. 2005), but it is unclear what effects the chemicals will exert on the reprogramming process. The optimal scenario calls for an endosomolytic domain to be present in the N-terminal R9 fusion peptide. Endosomal escape via fusion partners appears possible. For example, fusogenic influenza HA2 facilitated endosomal escape for its fusion cargoes (Wadia et al. 2004; Michiue et al. 2005).

ur findings demonstrate the feasibility of the non-viral fusion protein approach for iPSC generation. We have achieved the first step by developing CFPS reactions that enable the production of significant quantities of fusion R9-RFs. We encountered considerable proteolysis and solubility problems and addressed them to produce full-length and soluble fusion R9-RFs. These fusion R9-RFs exhibit specific binding to their consensus dsDNA sequences and translocate across the plasma membranes in fibroblasts. R9-RF solubility as well as enhance R9-RF endosomal escape is enhanced so as to effectively and safely generate iPSCs using fusion protein RFs.

TABLE 1
List of plasmids used in this study.

| | | Native | Fusion |
|---|---|---|---|
| | | Protein MW | Protein MW |
| Plasmid Name | Gene Description | (kDa) | (kDa) |
| pET24a-R9-Nanog | CAT9-StrepTagII-Xa- | 34.6 | 39.7 |
| | R9-His₆-G₄S-Nanog | | |
| pET24a-R9-Oct3/4 | CAT9-StrepTagII-Xa- | 38.4 | 43.5 |
| | R9-His₆-G₄S-Oct3/4 | | |
| pET24a-R9-Sox2 | CAT9-StrepTagII-Xa- | 34.3 | 39.4 |
| | R9-His₆-G₄S-Sox2 | | |
| pET24a-R9-Lin28 | CAT9-StrepTagII-Xa- | 22.7 | 27.8 |
| | R9-His₆-G₄S-Lin28 | | |
| pET24a-R9-c-Myc | CAT9-StrepTagII-Xa- | 48.8 | 53.9 |
| | R9-His₆-G₄S-c-Myc | | |
| pET24a-R9-Klf4 | CAT9-StrepTagII-Xa- | 50.1 | 55.2 |
| | R9-His₆-G₄S-Klf4 | | |

Native MW refers to the molecular weight of the wild-type protein. Fusion MW refers to the molecular weight of the human R9 fusion reprogramming factors with the 5.1 kDa N-terminal R9 fusion peptide and the desired additional conjugates. (See FIG. 1 legend for definitions of abbreviations used in the gene descriptions).

### Example 2

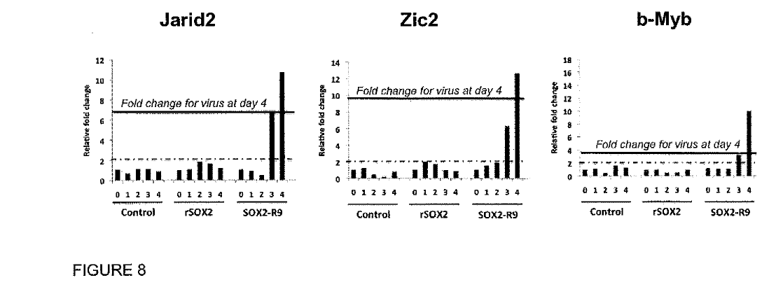

This example is offered to demonstrate that the transcription factors produced by methods provided by this invention are fully capable of producing the desired biological effects, the stimulation of nuclear expression from the naturally targeted genes. In order to affect gene expression, the fusion protein must enter the cell, reach the nucleus, and activate the genes normally activated by the transcription factor. For this example, the R9-Sox2 fusion protein was produced by the cell-free protein synthesis (CFPS) methods described in Example 1. The fusion protein was then purified and added to the cell growth medium at a 200 nM concentration each day for three days. The data presented in FIG. 8 indicate that the Sox2 regulated genes, Jarid2, Zic2, and b-Myb, each respond by expressing their transcription product at the same or higher level as that stimulated by retroviral infection of a vector that expresses the Sox2 transcription factor in vivo. The latter is now considered as the “gold standard” comparison. In contrast, recombinant Sox2 (rSox2) is not active because it lacks the R9 fusion partner required to stimulate cell entry.

### Materials and Methods

Culturing the target cells and preparing the positive control. The human neonatal foreskin BJ fibroblast cell line (passage ˜6) was cultured in DMEM with 10% FBS and 1% penicillin/streptomycin (pen-strep) antibiotics (Invitrogen) in a humidified 5% CO₂ incubator at 37° C. To provide the positive control culture, the cDNA for Sox2 was cloned into the retroviral pMX vector and separately transfected into 293FT cells using lipofectamine 2000 (Invitrogen). Viral supernatants were harvested 3 days later, concentrated, and used to infect human BJ fibroblasts grown in DMEM medium containing 10% FBS and 1% pen/strep. To test the protein nuclear reprogramming factors, BJ fibroblast cells were grown to 80% confluency and were then serum-starved using DMEM medium with 1% serum to induce G1 cell cycle arrest. The synchronized BJ fibroblasts were then treated with 100 nM R9-Sox2 fusion protein or 100 nM rSox2 at 0, 24, and 48 hours.

Determining nuclear transcriptional responses from Sox2 regulated genes. Cellular RNA was extracted for real-time RT-PCR analysis at 0, 24, 48, and 72 hours after the initial addition of the transcription factors or after retroviral infection. Cultured BJ fibroblasts were collected using TrypLE EXPRESS (Invitrogen) and treated with TRIzol® (Invitrogen). Cellular RNA was purified using an RNeasy Mini Kit (QIAGEN) according to the manufacturer's recommendations. Purified RNA was then treated by DNase I (QIAGEN) to remove genomic DNA contamination. First-strand cDNA synthesis was performed with 2 μg total RNA for each sample in a total volume of 20 μl. The reverse transcription reaction was performed with random primers and incubated at 25° C. for 10 min followed by 42° C. for 50 min. Real-time RT-PCR analysis of Jarid2, Zic2, and b-Myb mRNA was performed using Gene Expression Assays with Taqman assay primers (Applied Biosystems, Foster City, Calif.). Analysis of 18S mRNA served as an internal control. The TaqMan assay IDs are as follows: Jarid2 assay ID: Hs01004457_ml, Zic2 assay ID: Hs00600845_ml, b-Myb assay ID: Hs00193527_ml, and 18S assay ID: Hs99999901_s1. All PCR reactions were performed in a total volume of 20 μl containing diluted 2× TaqMan Universal PCR Master Mix (Applied Biosystems) and 20× Gene Expression Assay Mix and 40 ng cDNA. All assays were performed in duplicate and run on an 7300 ABI Real time PCR System using the following conditions: 50° C. for 2 min, 95° C. for 10 min, and 40 cycles of 95° C. for 15 sec and 60° C. for 1 min. Relative quantification of the amplified products was based upon Ct values.

### Example 3

This example is offered to illustrate how the methods offered by this invention facilitate convenient and rapid experimentation for improving the expression level and solubility of the nuclear reprogramming factors. The transcription factor, Oct4, was chosen since initial results suggested that it was difficult to produce in a soluble form. The accessibility to the protein translation and folding compartment that is provided by CFPS was used to survey a wide variety of chemical environments. Different ions from the Hofineister series were evaluated since they affect water activity to different degrees and this might be expected to affect protein folding. In addition, representative detergents were evaluated as these might also influence protein expression and folding by interacting with hydrophobic sequences within the protein. CFPS was performed as described in Example 1 except that individual PANoX SP reactions were incubated for 3 hours in wells in a 96-well plate. Total and soluble Oct4 accumulation were determined using ¹⁴C-leucine incorporation as also described.

A Resolution IV fractional factorial experiment was designed and analyzed using Stat-Ease DesignExpert software (Minneapolis, Minn.) with the following variables: potassium glutamate (175 mM or 350 mM), temperature (room temperature or 30° C.), n-dodecyl beta-D-maltoside (DDM) detergent addition (0% or 0.1%), Tween20 detergent addition (0% or 0.1%), and the addition of ammonium sulfate, potassium nitrate, and/or potassium oxalate (0 mM or 50 mM).

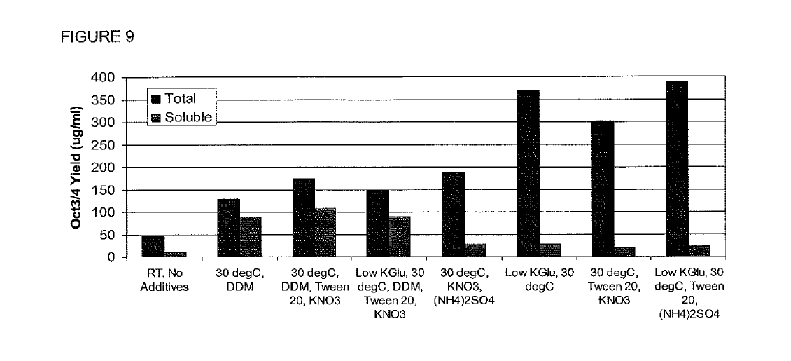

Selected data are presented in FIG. 9. The experiment did not identify any statistically significant synergies or two-factor interactions, but did identify beneficial one-factor effects. Halving the concentration of potassium glutamate in the CFPS reaction from 350 mM to 175 mM improves total protein yields and performing CFPS in the presence of 0.1% n-dodecyl beta-D-maltoside (DDM) detergent dramatically improves soluble protein yields. Cooperative effects such as a possible synergy between DDM, Tween20, and potassium nitrate addition are suggested. The use of such convenient, iterative experimentation can readily be used to optimize the production of soluble, active reprogramming factors.

What is claimed is:

1. A method of producing a composition of reprogramming transcription factors (RF); the method comprising:
synthesizing one or more reprogramming transcription factors in a cell-free reaction mixture.

2. The method of claim 1, wherein the reprogramming transcription factor comprises a permeant domain.

3. The method of claim 2, wherein the permeant domain comprises a plurality of arginine residues.

4. The method of claim 3, wherein the reprogramming factor is selected from Oct3/4; Sox2; Klf4; c-Myc; Nanog, and Lin28

5. The method of claim 2, wherein said reprogramming transcription factor further comprises a fusion partner that enhances solubility, and/or a partner that provides endosomolytic activity.

6. The method of claim 1, wherein said cell-free synthetic reaction is performed at about 25° C.

7. The method of claim 1, wherein said cell-free synthetic reaction utilizes a bacterial cell extract from a cell that is genetically altered to have decreased endogenous protease activity

8. The method of claim 1, further comprising a step of increasing solubility with on column refolding.
